(19) United States
(12) Patent Application Publication
(10) Pub. No.: US 2023/0015541 A1
(43) Pub. Date: Jan. 19, 2023

(54) 16% Sodium Citrate Solution for Use as an Antimicrobial Catheter Lock Solution

(71) Applicant: Becton, Dickinson and Company, Franklin Lakes, NJ (US)

(72) Inventor: Cristian Vilorio, Jersey City, NJ (US)

(21) Appl. No.: 17/867,211

(22) Filed: Jul. 18, 2022

Related U.S. Application Data
(60) Provisional application No. 63/223,191, filed on Jul. 19, 2021.

(51) Int. Cl. A61L 29/16; A01N 37/36; A01N 25/02

(52) U.S. Cl. CPC A61L 29/16; A01N 37/36; A01N 25/02; A61L 2202/24

(57) ABSTRACT

Provided herein are catheter lock solutions having anticoagulation and antimicrobial properties, the catheter lock solutions including citrate salts. The citrate salt can be trisodium citrate, and the catheter lock solution can further include a diluted acid for adjusting the pH of the catheter lock solution. In one configuration, the lock solution is between 15.5% and 16.5% w/v of a citrate salt, water for injection, and optionally an acid. The pH of the catheter lock solution may be between 6.4 and 7.5.

### CROSS-REFERENCE TO RELATED APPLICATION

The present application claims priority to U.S. Provisional Application Ser. No. 63/223,191, filed Jul. 19, 2021, entitled “16% Sodium Citrate Solution for Use as an Antimicrobial Catheter Lock Solution”, the entire disclosure of which is hereby incorporated by reference in its' entirety.

### BACKGROUND OF THE INVENTION

### Field of the Invention

The present invention relates to the maintenance of catheters in a condition that is substantially free of blood and clots. More particularly, the invention relates to the use of a solution of citrate salt to prevent backflow and maintain patency in a catheter lumen.

### Description of Related Art

Catheters, particularly intravenous (IV) catheters, may be used for infusing fluid, such as a medication, into a patient or withdrawing fluid, such as blood, from a patient. Catheters may include a lumen or reservoir which contains fluid or medication to be injected into, or removed from, a patient's body. In certain configurations an injection port may be provided with the catheter.

Complications associated with catheters include thrombosis, infection, and clotting. Catheter occlusions will often occur due to thrombotic complications related to the formation of a fibrin sheath within the lumen or at the tip of the catheter. Formation of a fibrin sheath may allow for adherence of bacteria to the interior of the catheter lumen and serve as a locus for catheter related infection.

To reduce problems associated with clotting and thrombus formation, it is common to “lock” intravascular access catheters between successive uses. Locking typically involves first flushing the catheter with saline to remove blood and other substances from the catheter lumen. After the catheter has been flushed, an anti-coagulant solution, typically heparin, is then injected to displace the saline and fill the lumen. The heparin locking solution prevents blood from entering the lumen and actively inhibits clotting and thrombus formation within the lumen.

The heparin lock solution is infused into the catheter lumen immediately after each use, and is left within the catheter until the catheter is accessed again. The heparin lock solution must be withdrawn from the catheter before the next use so that the heparin is not introduced into the body of the patient. In some instances, heparin lock solutions include up to 10,000 units of heparin per catheter lumen. Infusing this amount of heparin into a patient may result in excessive bleeding.

However, even with the use of a traditional heparin lock solution, the catheter can become occluded between uses from coagulation of blood within the catheter. Blood may be present within the catheter because an inadequate volume of heparin was infused within the catheter lumen, the heparin lock solution diffused from the lumen, or residual blood remains in the lumen. This can result in formation of a thrombus with concomitant loss of patency and decreased flow through the catheter lumen.

There remains a need for a catheter lock solution which can provide long-lasting action and increased safety, without the need for additional applications in between uses of the catheter.

### SUMMARY OF THE INVENTION

Accordingly, provided herein is a catheter lock solution that includes a citrate salt. In aspects the citrate salt is a sodium citrate salt. In further aspects, the citrate salt is trisodium citrate.

In aspects, the catheter lock solution further includes an acid. In aspects, the acid is a diluted acid. In some aspects, the diluted acid is diluted hydrochloric acid (HCl).

In aspects, the catheter lock solution includes the citrate salt in an amount between about 15.5% and 16.5% w/v.

In aspects, the lock solution includes the acid in an amount between about 0% and about 0.7% v/v.

In certain aspects, the catheter lock solution includes 16% w/v trisodium citrate, water-for-injection (WFI), and an amount of 10% HCl sufficient to maintain a pH of the lock solution at between about 6.4 and about 7.5.

In certain aspects, the catheter lock solution is free of any additional component having anticoagulant or antimicrobial activity.

Also provided herein is a method of making a catheter lock solution. The method includes the steps of dissolving a citrate salt, preferably trisodium citrate, in WFI and adding a diluted acid, preferably 10% HCl, until the pH of the catheter lock solution is between about 6.4 and about 7.5.

Also provided herein is a catheter lock solution including only trisodium citrate, water-for-injection (WFI), and 10% HCl.

In aspects, the catheter lock solution includes only between about 15.5% and about 16.5% w/v trisodium citrate, WFI, and an amount of 10% HCl sufficient to maintain a pH of the lock solution at between about 6.4 and about 7.5.

Also provided herein is a method of making a catheter lock solution. The method includes only the steps of dissolving trisodium citrate in WFI and adding 10% HCl until the pH of the catheter lock solution is between about 6.4 and about 7.5.

Also provided herein is a catheter lock solution including only about 16% w/v trisodium citrate, water-for-injection (WFI), and an amount of 10% HCl sufficient to maintain a pH of the lock solution at about 7.

Also provided herein are pre-filled syringes including syringes containing a catheter lock solution as described herein.

Also provided herein are catheters including a tube defining a lumen therethrough, at least a portion of the lumen being infused with a catheter lock solution as described herein.

Also provided herein is a method of inhibiting coagulation and microbial activity in a catheter including the steps of providing a catheter including a tube defining a lumen therethrough and infusing into at least a portion of the lumen the catheter lock solution described herein.

### BRIEF DESCRIPTION OF THE DRAWINGS

FIG. 1 is a longitudinal cross-section of a pre-filled syringe including the catheter lock solution according to one aspect of the lock solution described herein.

FIGS. 2A and 2B are tables showing minimum inhibitory concentration (MIC) of sodium citrate for numerous microorganism strains.

FIG. 3 is a table showing growth inhibition timeframe of Gram-positive Staphylococcus aureus exposed to various concentrations of sodium citrate.

FIG. 4 is a table showing growth inhibition timeframe of Gram-negative Pseudomonas aeruginosa exposed to various concentrations of sodium citrate.

FIG. 5 is a table showing growth inhibition timeframe of the fungus Candida albicans exposed to various concentrations of sodium citrate.

FIG. 6 is a table showing effects of 4% sodium citrate on coagulation across various time points.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

Provided herein is a catheter lock solution including a citrate salt, a solvent, and a diluted acid. The lock solution described herein provides patency for a catheter and exhibits anticoagulation and antibiotic activity. Without wishing to be bound by the theory, it is believed that the citrate salt acts as an anticoagulant by chelating calcium (Ca²⁺), in blood. Such metal ions are necessary for proper functioning of various coagulation cascades, and, for example in the case of Ca²⁺, coagulation Factor V Platelets, Factor VII Platelets, Factors IX to IXa, and/or Factors XI to XIa. By chelating calcium (Ca²⁺), the cascade is blocked and coagulation is inhibited. With regard to antimicrobial activity, and again not wishing to be bound by the theory, it is believed that the citrate salt chelates Ca²⁺, magnesium (Mg²⁺), zinc (Zn²⁺), and/or copper (Cu²⁺), which are necessary for the function of certain enzymes required for bacterial and fungal proliferation and survival. A chelating agent such as a citrate salt will bind to the aforementioned pool of elements at varying degrees.

The lock solution and pre-filled syringe described herein also minimize syringe-induced reflux of blood into an implanted catheter. The lock solution described herein provides significant advantages over typical heparin-based locks, in that adverse events related to use of heparin, such as heparin-induced thrombocytopenia, systemic bleeding complications, and assay interference, can be avoided. In addition, a citrate salt-based lock offers significant cost and time savings over heparin-based solutions. A lock solution as described herein also offers advantages in terms of stable shelf-life, such as at least two years from time of manufacture. Moreover, the use of a pre-filled syringe saves time, improves sterility, and thus safety, of the product and delivery thereof, and eliminates the likelihood of contamination that can occur during manual filling.

As used herein, the term “lock solution” or “locking solution” refers to a solution that is injected or otherwise infused into a lumen of a catheter with the intention of allowing a substantial portion of the solution to remain in the lumen until it is desired or required to access or re-access the lumen, typically for additional treatment or maintenance. Additional treatment may include, for example, infusion or withdrawal of fluid into/from the lumen of a catheter. The locking solution may be placed into the catheter to provide short or long-term protection. Preferably, the lock solution can remain in the lumen for a desired amount of time lasting up to about one week, and in aspects up to about a month. However, the lock solution may be changed on a daily basis, such as during regular care or sterile maintenance of the catheter. The catheter may be changed or refreshed by aspirating the lock solution out of the catheter lumen, and locking the catheter with new catheter lock solution within the catheter for a desired amount of time. Use of a lock solution described herein may prolong the lifetime of the catheter, lengthen the interval between required replacements of the lock solution, and/or inhibit infection in a patient.

The term “catheter” as used herein refers to a tube defining a lumen therethrough that may be inserted into part of the body or provided in communication with a body or other biological culture to deliver a fluid thereto or remove a fluid therefrom. In aspects, the catheter lock solution as described herein may be used to provide anticoagulant activity (inhibit coagulation) and antimicrobial activity in a catheter, such as a soft catheter or a hard catheter.

As used herein, the term “anticoagulant activity” refers to inhibition or prevention of blood coagulation.

As used herein, the term “antimicrobial activity” refers to destruction, inhibition, or prevention of the propagation, growth, or multiplication of unwanted micro-organisms, such as aerobic and anaerobic Gram-positive and Gram-negative bacteria, undulating bacteria, spirochetes, spores, spore-forming micro-organisms, yeasts, fungi, molds, viruses, aerobic organisms, anaerobic organisms, and mycobacteria. Without limitation, the catheter lock solution as described herein can have antimicrobial activity against Gram-positive bacteria such as Staphylococcus aureus ATCC 33591, Staphylococcus epidermidis ATCC 12228, and Enterococcus faecalis ATCC 29212, Gram-negative bacteria such as Pseudomonas aeruginosa ATCC 27853, Escherichia coli ATCC 25922 and Klebsiella pneumoniae ATCC 13883, and fungi such as Candida albicans ATCC 10231, and Candida auris AR #00383 for up to 24 hours or longer.

The catheter lock solution as described herein can be used to inhibit microbial activity and coagulation of catheters that are placed into particular parts of the body to allow, for example, drainage of urine from the urinary bladder as in urinary catheterization; drainage of fluid collections; administration of intravenous fluids, medication, or prenatal nutrition; angioplasty; angiography; balloon septostomy; and direct measurement of blood pressure in an artery or vein. While the catheter lock solution as described herein may be used to inhibit microbial activity and coagulation of any catheter, the catheter lock solution may be used to inhibit microbial activity and coagulation of catheters that are used, for example, for hemodialysis and hemofiltration that rely on separate draw and return catheters implanted into a vein to allow extracorporeal treatment of the blood or for peritoneal dialysis, which relies on a single catheter implanted in the peritoneum to permit introduction and withdrawal of dialysate to permit in situ dialysis.

By preventing coagulation within the lumen of a catheter, the citrate salt lock solution as described herein maintains patency within the catheter. As used herein, the term “patency” refers to a catheter being open or unobstructed, for example by clots or fibrin sheaths within the lumen of the catheter.

As described above, the lock solution includes a citrate salt. As used herein, the term “citrate salt” refers to a salt of citric acid. Citric acid is a tricarboxylic acid having the formula C6H807, and is considered a weak acid. Examples of suitable citrate salts for use in the lock solution described herein are sodium salts and potassium salts. In aspects, the citrate salt is monosodium, disodium, or trisodium citrate. In particular aspects, the citrate salt is trisodium citrate. In some aspects, the trisodium citrate is trisodium citrate di-hydrate, a powder form of trisodium citrate that can be dissolved in a solvent.

As described above, it is believed that trisodium citrate functions as an anticoagulant by chelating metal ions, such as Ca²⁺ ions in the blood, disrupting the coagulation cascade. As a lock solution, trisodium citrate acts in two ways to maintain catheter patency. First, the physical presence of the lock solution prevents backflow of blood from the patient into the catheter lumen, reducing the risk of clotting or occlusion of the lumen. Second, to the extent that any blood does backflow into the catheter lumen, by disrupting the coagulation cascade the trisodium citrate prevents clotting in the catheter lumen and at the catheter tip.

The lock solution includes a solvent in which the citrate salt is dissolved. The solvent can be any biocompatible solvent. In aspects, the solvent is water-for-injection (WFI). In aspects, the citrate salt is included in the solvent at a mass per volume concentration of between about 15.5% and about 16.5% weight per volume (w/v), all subranges and values therebetween inclusive. As used herein, the term “about” refers to a difference of ±10% of the value. In aspects, the lock solution is made by dissolving trisodium citrate di-hydrate at a mass per volume (w/v) of about 16% in WFI. In non-limiting embodiments or aspects, the lock solution contains no more than about 16.5% trisodium citrate and no less than about 15.5% trisodium citrate.

Lock solutions of lower concentrations (e.g., 4% w/v) of a citrate salt, as well as lock solutions of higher concentrations (e.g, 30% w/v and 46.7% w/v), are known. A lock solution including about 16% w/v of a citrate salt has the benefit of potent antimicrobial effect, while reducing and/or eliminating potential adverse events associated with much higher concentrations of citrate salt, such as hypocalcemia, which can cause global anticoagulation. Further, the concentration of citrate salt described herein can lessen and/or eliminate the need to use heparin, which has no antimicrobial properties, and the use of which can lead to heparin-induced thrombocytopenia and systemic hepranization/systemic anticoagulation.

The lock solution may also include a diluted acid to adjust the pH of the solution. The diluted acid can be any biocompatible organic or inorganic acid. In aspects, the diluted acid is 10% hydrochloric acid (HCl). In aspects, the lock solution includes between about 0 and about 0.7% v/v of diluted acid, all subranges and values therebetween inclusive. In aspects, the lock solution includes between about 0 and about 0.7% v/v of 10% HCl. In aspects, the lock solution includes about 0.11% v/v of acid, in aspects HCl. In non-limiting embodiments or aspects, the lock solution includes an amount of acid, optionally diluted acid, optionally 10% HCl, sufficient to maintain a pH of the lock solution in a range of about 6.4 to about 7.5. Those of skill in the art will understand that the amount of diluted acid can be adjusted to achieve a preferred pH of the lock solution of between about 6.4 and about 7.5.

In particular aspects of the lock solution described herein, the solution includes between about 15.5% and about 16.5% w/v of trisodium citrate in WFI, and the lock solution has a pH of between about 6.4 and about 7.5, and in some aspects between about 6.54 and about 7.25, all subranges therebetween inclusive. In some aspects, the pH of the lock solution when delivered to a syringe during preparation of pre-filled syringes is between about 6.57 and about 7.16, all subranges therebetween inclusive. In some aspects, the pH of the lock solution when delivered to a syringe during preparation of pre-filled syringes is about 6.87.

In aspects, between about 0 and about 0.7% v/v of 10% HCl, optionally 0.11% v/v is included in the lock solution. In some aspects, the lock solution includes about 16% w/v trisodium citrate di-hydrate in WFI, and sufficient diluted (e.g., 10%) HCl to provide a lock solution with a pH of about 6.9. In some aspects, the lock solution includes about 16% w/v trisodium citrate in WFI and 0.11% v/v of diluted (e.g., 10%) HCl to provide a lock solution with a pH of 7. In further aspects, the lock solution includes trisodium citrate, WFI, and, optionally, HCl to provide the required pH, and includes no additional anticoagulant or antimicrobial additives. In aspects the catheter lock solution is free of excipients. In some aspects the catheter lock solution is free of alcohols, glycerol, polyethylene glycols, citric acid, and/or polysorbate. In aspects the catheter lock solution is free of any component other than a citrate salt, WFI, and, optionally, HCl.

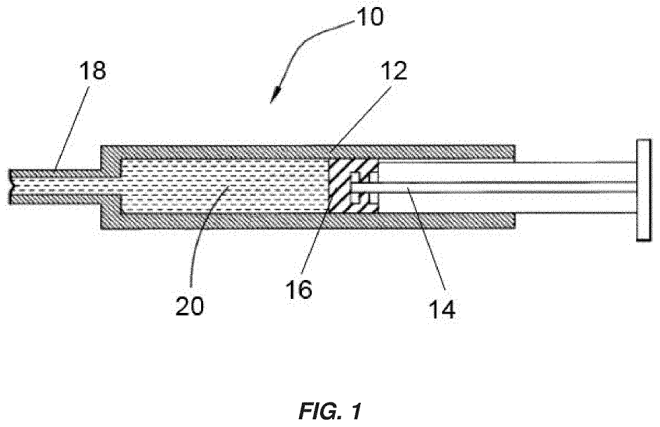

Also provided herein is an infusion device containing the lock solution as described above. In aspects, the infusion device is a pre-filled syringe including the lock solution as described above. In aspects, the pre-filled syringe includes a distal end, a proximal end, and a barrel therebetween defining a reservoir. The pre-filled syringe includes a plunger at the proximal end and a connector at the distal end configured to connect to a catheter, other needle-free connectors, Y-sites, and the like. In aspects, the connector at the distal end of the pre-filled syringe is a male or female luer connector. With reference to FIG. 1, illustrated is a pre-filled syringe 10 containing a catheter lock solution 20, as described above. The pre-filled syringe 10 includes a barrel 12, plunger rod 14, stopper 16, and luer connection 18. In some aspects, the pre-filled syringe 10 is formed of polypropylene. In particular aspects, one or more of the barrel 12, plunger rod 14, and tip cap (not illustrated) are formed of polypropylene. In aspects, the stopper 16 of the pre-filled syringe 10 is an elastomeric stopper.

In aspects, the pre-filled syringe 10 is designed or configured to reduce or prevent instances of reflux of blood into a catheter at the conclusion of flushing with a lock solution as described herein. In aspects, the pre-filled syringe 10 is configured such that the plunger rod 14 is shorter than a typical plunger rod, such that compression of the stopper 16 following infusion of the lock solution 20 is substantially or completely prevented. In other aspects, the stopper 16 is designed or configured such that the nose of the stopper comes into contact with a distal end of the barrel, adjacent the luer 18, and blocks the opening, preventing vacuum and thus reflux of blood into the catheter.

Also provided herein is a method of locking a catheter including the steps of infusing the lock solution as described above into a catheter lumen. In aspects, the method further includes the step of flushing the catheter lumen prior to infusing the lock solution.

In aspects, the method includes the steps of providing a catheter having an interior surface and an exterior surface, and infusing into at least a portion of the interior surface with the catheter locking solution. Preferably, the locking solution is infused into the interior surface such that the interior surface is substantially filled. Non-limiting examples of interior surfaces of the catheter that can be filled with the catheter locking solution described herein include the lumen, related tubing, plungers, caps, and extension sets. Other devices capable of being coated or filled with the catheter locking solution described herein include the inner lumen of vascular access devices, as well as, needle-less access devices. The locking solution can be infused by any conventional method well known to those skilled in the art, such as dipping, spraying, or injecting, for example and without limitation, using the pre-filled syringe as described herein.

When the lock solution described above is infused into the interior surface of the catheter, a sufficient amount of the lock solution can be injected to fill or substantially fill the interior volume/space of the catheter, as well as, any adjacent surfaces or lumens of any attached access device. Alternatively, a volume less than the amount of fluid needed to fill the catheter can be infused into the interior surface. For example, a sufficient amount of lock solution can be infused into the catheter to fill, for example, from 80% to 250% of the internal volume of the catheter, all subranges and percentages therebetween inclusive. In yet another aspect, an amount greater than the internal volume of the catheter can be infused. For example, an amount of the lock solution greater than the internal volume of the catheter can be infused into the lumen. Unlike heparin-based lock solutions, this overflow can be utilized without adverse effects on the clotting system of the patient. The lock solution may be infused or flushed into the catheter between 1 and 1000 times, all subranges and values therebetween inclusive.

The method of locking a catheter, as described above, is effective to prevent backflow of blood from the patient into the lumen of the catheter into which the lock solution is infused or introduced. In addition, the lock solution reduces the occurrence of, or prevents clot formation, maintaining catheter patency.

Also provided herein is a method of making a catheter lock solution as described above. The catheter locking solution can be prepared with simple mixing of the above-identified components at room temperature to provide anticoagulation and antimicrobial activity. In other aspects, the solution is prepared in bulk and loaded into syringes to prepare pre-filled syringes that can be distributed and stored until needed.

Also provided herein is a catheter including a tube defining a lumen therethrough that is pre-filled with the catheter locking solution described above prior to insertion within the patient.

### EXAMPLES

### Example 1

In vitro testing was conducted with sodium citrate on samples of various microorganisms to determine a minimum inhibitory concentration (MIC) of sodium citrate (at pH 6.4-7.5) for those microorganisms. As used herein, “MIC” means the lowest concentration of sodium citrate (% w/v) that visibly inhibits microbial growth. Testing was performed as set forth in ‘Methods for Dilution Antimicrobial Susceptibility Tests for Bacteria That Grow Aerobically,’ 11^{th} Edition, Clinical and Laboratory Standards Institute (2018), ‘Methods for Determining Bactericidal Activity of Antimicrobial Agents' Approved Guideline.’ Vol. 12, No. 9, Clinical and Laboratory Standards Institute (1999), and ‘Development of In Vitro Susceptibility Testing Criteria and Quality Control Parameters; Approved Guideline-Second Edition,’ Vol. 21, No. 7, Clinical and Laboratory Standards Institute (2001).

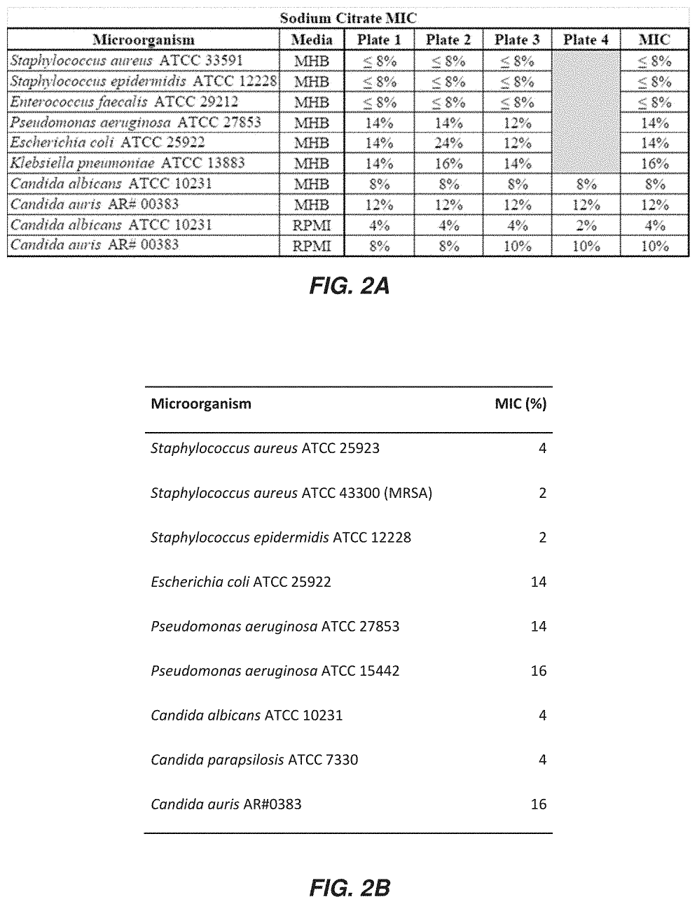

For MIC determinations, sodium citrate was reconstituted and diluted to various working stock concentrations (8%, 10%, 12%, 14%, 16%, 20%, 24%, and 28% in deionized water and transferred into round-bottom 96-well microdilution plates. Three strains each of Gram-positive bacteria, Gram-negative bacteria, and yeast were obtained from the American Tissue Type Collection (ATCC) or the Centers for Disease Control Antibiotic Resistant (CDC AR) Isolate Bank. Cultures were grown on Trypticase Soy Agar (TSA, for bacteria) or Sabouraud Dextrose Agar (SDA, for yeast) and suspended in Mueller Hinton Broth to a final cell density of approximately 5×10⁵ CFU/mL during exposure to sodium citrate. Following incubation at 35±2° C. for 24±2 h (bacteria) or 44±4 h (yeast), microdilution plates were visually examined for the presence or inhibition of growth. Trials were duplicated and the results are shown in FIGS. 2A and 2B.

As will be appreciated, the MIC is shown, thus testing of higher concentrations was deemed unnecessary, because if, for example 4% sodium citrate is the MIC for a given microorganism, then 16% would be at least as effective. Three (3) replicates were run for each bacteria evaluated and four (4) replicates were run for each fungi evaluated, using two (2) different growth media for the fungi. The number of replicates ensure confidence when identifying the MIC of aqueous sodium citrate for the aforementioned microorganisms. As illustrated in FIGS. 2A and 2B, 16% w/v sodium citrate will prevent growth and proliferation of Gram-negative and Gram-positive bacteria, as well as yeast.

### Example 2

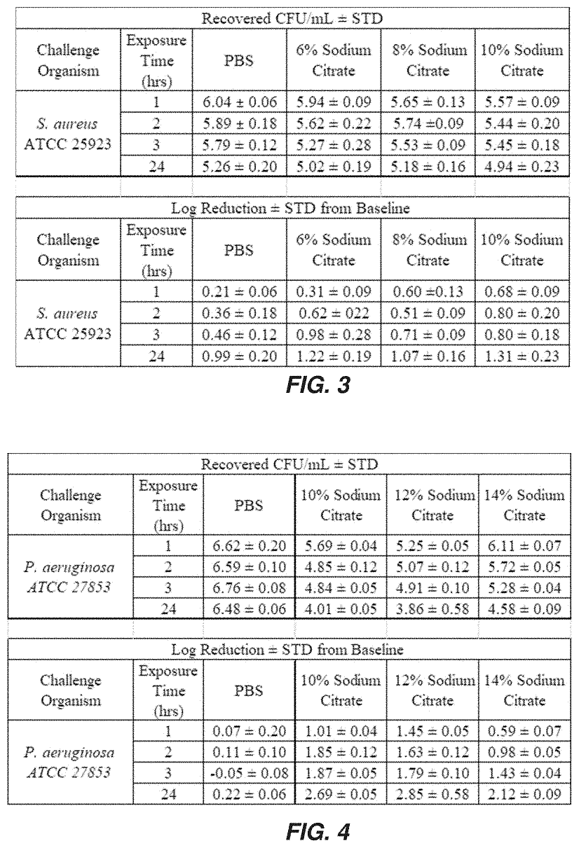

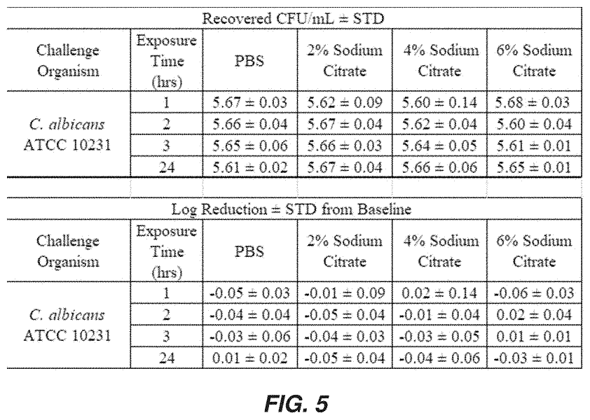

In vitro testing was conducted with various concentrations of sodium citrate on S. aureus (Gram-positive bacteria) (FIG. 3), P. aeruginosa (Gram-negative bacteria) (FIG. 4), and yeast, C. albicans (FIG. 5). Testing was conducted as set forth in ‘Methods for Dilution Antimicrobial Susceptibility Tests for Bacteria That Grow Aerobically,’ 11^{th} Edition, Clinical and Laboratory Standards Institute (2018), ‘Methods for Determining Bactericidal Activity of Antimicrobial Agents' Approved Guideline.’ Vol. 12, No. 9, Clinical and Laboratory Standards Institute (1999), and ‘Development of In Vitro Susceptibility Testing Criteria and Quality Control Parameters; Approved Guideline-Second Edition,’ Vol. 21, No. 7, Clinical and Laboratory Standards Institute (2001). Briefly, trisodium citrate salt was compounded using purified water and the pH was adjusted, if necessary, with hydrochloric acid to provide a composition with a pH between 6.4 and 7.5. With regard to FIG. 3, the data shows that through 24 hours, at concentrations of 6% (w/v), 8% (w/v) and 10% (w/v) sodium citrate, S. aureus does not grow, and, in fact, a decrease in bacteria count is seen. Thus, it is expected that 16% (w/v) trisodium/sodium citrate will prevent the growth of S. aureus and similar Gram-positive bacteria for up to 24 hours. The data suggests that the longer the bacteria is exposed to the aqueous sodium citrate, the likelihood that the bacteria count will decrease. Due to this, it is expected that growth inhibition and possibly eradication can occur beyond 24 hours.

With regard to FIG. 4, the data shows that through 24 hours, at concentrations of 10% (w/v), 12% (w/v), and 14% (w/v) sodium citrate, P. aeruginosa does not grow, and, in fact, a decrease in bacteria count is seen. Thus, it is expected that 16% (w/v) trisodium/sodium citrate will prevent the growth of P. aeruginosa and similar Gram-negative bacteria for up to 24 hours. The data suggests that the longer the bacteria is exposed to the aqueous sodium citrate, the likelihood that the bacteria count will decrease. Due to this, it is expected that growth inhibition and possibly eradication can occur beyond 24 hours.

With regard to FIG. 5, the data shows that through 24 hours, at concentrations of 2% (w/v), 4% (w/v), and 6% (w/v) sodium citrate, C. albicans does not grow. Thus, it is expected that 16% (w/v) trisodium/sodium citrate will prevent the growth of C. albicans and similar yeast for up to 24 hours.

### Example 3

The anticoagulation effect of a 4% sodium citrate lock solution was measured in vitro as activated clotting time (ACT) using whole human blood over 2 years. The anticoagulation effectiveness of 4% sodium citrate lock solution on whole blood was determined using the Sonoclot® Coagulation & Platelet Function Analyzer (Sienco Inc., Boulder Colo.), which can calculate the onset of clot formation by monitoring mechanical changes that occur in blood samples during hemostasis. The mechanism is a tubular probe that moves up and down within a blood sample. As the sample progresses through various stages of clotting, electronic circuitry (a transducer) detects increasing resistance. This produces a series of electronic signals that are processed by a microcomputer. The output is the total time of clot formation where the blood is more viscous than initially introduced.

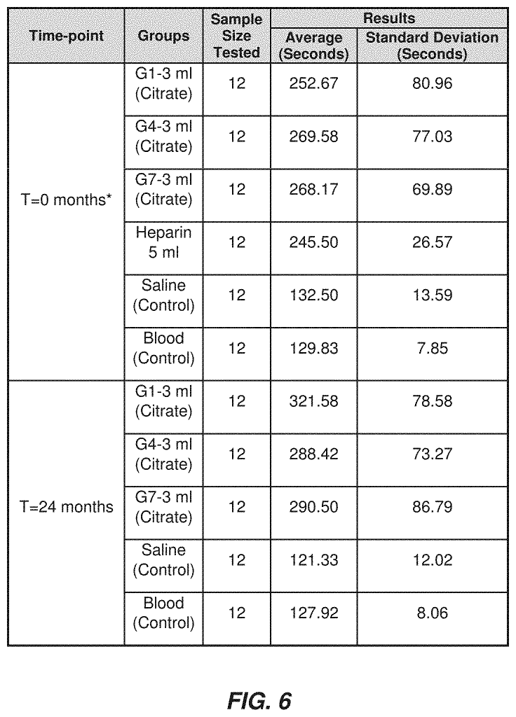

The data presented in FIG. 6 demonstrates that 4% (w/v) trisodium sodium citrate has anticoagulation efficacy as the time required for clot formation was longer relative to the controls (no solution and saline). Thus at 16% (w/v) trisodium sodium citrate, one would expect a more profound effect due to the greater concentration and the amount of citrate available to chelate calcium, thereby prolonging the coagulation cascade.

While the present invention has been described in terms of the above detailed description, those of ordinary skill in the art will understand that alterations may be made within the spirit of the invention. Accordingly, the above should not be considered limiting, and the scope of the invention is defined by the appended claims.

What is claimed is:

1. An antimicrobial catheter lock solution comprising:
about 15.5% w/v to about 16.5% w/v of a citrate salt;
water for injection; and, optionally,
an acid,
wherein a pH of the catheter lock solution is between about 6.4 and about 7.5, and
wherein the catheter lock solution is free of any additional components having anticoagulant or antimicrobial activity.

2. The antimicrobial catheter lock solution of claim 1, wherein the citrate salt is a sodium citrate salt.

3. The antimicrobial catheter lock solution of claim 1, wherein the citrate salt is trisodium citrate.

4. The antimicrobial catheter lock solution of claim 1, wherein the catheter lock solution further comprises an acid.

5. The antimicrobial catheter lock solution of claim 4, wherein the acid is a diluted acid.

6. The antimicrobial catheter lock solution of claim 5, wherein the diluted acid is diluted hydrochloric acid (HCl).

7. The antimicrobial catheter lock solution of claim 6, wherein the catheter lock solution comprises the acid in an amount between about 0.1% and about 0.7% v/v.

8. The antimicrobial catheter lock solution of claim 1, wherein the citrate salt comprises trisodium citrate di-hydrate.

9. The antimicrobial catheter lock solution of claim 1, wherein the catheter lock solution comprises about 16% w/v trisodium citrate, water-for-injection (WFI), and about 0.7% v/v of 10% HCl.

10. The antimicrobial catheter lock solution of claim 1, wherein the catheter lock solution is free of excipients.

11. The antimicrobial catheter lock solution of claim 1, wherein the catheter lock solution is free of paraben, alcohol, glycerol, polyethylene glycol, citric acid, and/or polysorbate.

12. A method of making the antimicrobial catheter lock solution of claim 1, consisting of dissolving a citrate salt in water-for-injection (WFI) and, optionally, adding an acid until the pH of the catheter lock solution is between about 6.4 and about 7.5.

13. A pre-filled syringe comprising a syringe containing the antimicrobial catheter lock solution of claim 1.

14. A catheter comprising a tube defining a lumen therethrough, wherein at least a portion of the lumen is infused with the antimicrobial catheter lock solution of claim 1.

15. A method of inhibiting coagulation and microbial activity in a catheter comprising:
providing a catheter comprising a tube defining a lumen therethrough; and
infusing, into at least a portion of the lumen of the catheter, the antimicrobial catheter lock solution of claim 1.

16. An antimicrobial catheter lock solution consisting of:
about 15.5% w/v to about 16.5% w/v of trisodium citrate;
water-for-injection (WFI); and
about 0.0% v/v to about 0.7% v/v of 10% HCl,
wherein the catheter lock solution has a pH of between about 6.4 and about 7.5.

17. An antimicrobial catheter lock solution consisting of:
about 16% w/v trisodium citrate;
water-for-injection (WFI); and
about 0.7% v/v of 10% HCl,
wherein the catheter lock solution has a pH of about 7.

18. The antimicrobial catheter lock solution of claim 17, wherein the 10% HCl is included in an amount of 0.11% v/v.
